(19) United States
(12) Patent Application Publication
(10) Pub. No.: US 2026/0234693 A1
(43) Pub. Date: Aug. 13, 2026

(54) HIGH-THROUGHPUT METHOD FOR SCREENING ALPHA-1,2-FUCOSYLTRANSFERASE AND SCREENED MUTANT

(71) Applicant: JIANGNAN UNIVERSITY, Wuxi (CN)

(72) Inventors: Long LIU, Wuxi (CN); Jian CHEN, Wuxi (CN); Xueqin LV, Wuxi (CN); Guocheng DU, Wuxi (CN); Xianhao XU, Wuxi (CN); Jianghua LI, Wuxi (CN); Yangeng LIU, Wuxi (CN); Yaokang WU, Wuxi (CN); Linhui PENG, Wuxi (CN)

(21) Appl. No.: 19/554,244

(22) Filed: Mar. 2, 2026

(30) Foreign Application Priority Data
Dec. 27, 2024 (CN) 202411949322.0

(51) Int. Cl. C12Q 1/48; C12N 1/20; C12N 15/72; C12P 19/44; C12Q 1/34; C12Q 1/6897; C12R 1/19

(52) U.S. Cl. CPC C12Q 1/48; C12N 1/20; C12N 15/72; C12P 19/44; C12Q 1/34; C12Q 1/6897; C12R 2001/19; G01N 2333/91102; G01N 2333/924

(57) ABSTRACT

The present invention provides a high-throughput method for screening α-1,2-fucosyltransferase and a screened mutant. The method includes the steps of constructing an indicator strain of 2′-fucosyllactose, so that the content of 2′-fucosyllactose is positively correlated with the fluorescence intensity, then expressing L-fucokinase and an α-1,2-fucosyltransferase mutant library in a chassis strain, embedding the chassis strain in water-in-oil droplets and fusing and co-culturing with the indicator strain, and obtaining a chassis strain comprising a forward mutant of α-1,2-fucosyltransferase by microfluidic sorting of droplets according to the fluorescence detection results. The enzyme activity of the mutant FutC^{V93I} is 2.31 times that of the original enzyme, so the enzyme activity is greatly enhanced. By using the method provided in the present invention, high-throughput screening of α-1,2-fucosyltransferase is achieved, which is beneficial to the industrial production, large-scale application and popularization of 2′-fucosyllactose in the fields of food, medicine, and others.

This application is a Continuation of PCT/CN2025/142360, filed on Dec. 15, 2025, which claims priority to Chinese Patent Application No. 202411949322.0, filed on Dec. 27, 2024, which is incorporated by reference for all purposes as if fully set forth herein.

A Sequence Listing XML file named “10015_0200.xml” created on Mar. 2, 2026, and having a size of 40,553 bytes, is filed concurrently with the specification. The sequence listing contained in the XML file is part of the specification and is herein incorporated by reference in its entirety.

### FIELD OF THE INVENTION

The present invention relates to the technical field of enzyme engineering, and in particular to a high-throughput method for screening α-1,2-fucosyltransferase and a screened mutant.

### DESCRIPTION OF THE RELATED ART

Fucosyltransferase (FucT) plays an important role in organisms, and is involved in many physiological and pathological processes, including inflammations, bacterial and viral infections, tumor metastasis and genetic disorders. The enzyme catalyzes the transfer of L-fucose from donor substrates to various sugar receptor substrates, including oligosaccharides, glycoproteins and glycolipids, thus affecting the functions of these biomolecules. In disease diagnosis and drug research and development, fucosyltransferase also show its importance. For example, in some patients with cancers, fucosyltransferase has a high activity, and the tumor diagnosis and therapeutic effect monitoring can be facilitated by detecting the activity of the enzyme. α-1,2-fucosyltransferase (FutC) plays an important role, because it can catalyze the synthesis of 2′-fucosyllactose (2′-FL), which is a compound having many physiological functions, including inhibiting infections with pathogenic bacteria, regulating intestinal flora and enhancing the immunity. 2′-FL has been approved for use as a nutritional ingredient in infant milk powder and special food. α-1,2-fucosyltransferase from Helicobacter pylori is most widely used in the biosynthesis of 2′-FL at present. However, the catalytic activity of the enzyme in a microbial expression system is low and the heterologous expression level is low, which limits the synthesis efficiency of 2′-FL.

To improve the catalytic performance and expression level of α-1,2 fucosyltransferase, directed evolution is an effective strategy. By directed evolution that simulates the Darwinian evolution process, numerous mutations are artificially produced by random mutations and recombination, and proteins with expected characteristics are screened by using selection stress selected according to specific needs and purposes. For example, the catalytic activity and thermal stability of α-1,2-fucosyltransferase from Helicobacter pylori can be improved by semi-rationally designed site saturation mutagenesis. This strategy is of great significance to the solution of the technical bottleneck associated with FutC and the industrial application of 2′-FL.

In the process of directed evolution, it is important to construct a high-throughput screening method. Currently, the researchers have developed many photochemical analysis techniques, based on the ultraviolet or fluorescence characteristics of substrates, products or their corresponding derivatives. These methods are widely used in enzyme engineering, metabolic engineering and other fields. However, no reasonable and efficient high-throughput method is available for screening high-activity α-1,2-fucosyltransferase, which limits the development of 2′-fucosyllactose biosynthesis. Therefore, it is very important to develop a new high-throughput screening method for discovering and improving α-1,2-fucosyltransferase, which will improve the production efficiency and reduce the cost of 2′-FL, and further promote its application in food and medicines.

### SUMMARY OF THE INVENTION

To solve the above technical problems, the present invention provides a high-throughput method for screening α-1,2-fucosyltransferase, which includes constructing an indicator strain of 2′-fucosyllactose and positively correlating the fluorescence intensity with 2′-fucosyllactose, expressing β-galactosidase, L-fucokinase and an α-1,2-fucosyltransferase mutant library in a chassis strain, embedding the chassis strain in water-in-oil droplets and co-culturing with the indicator strain, and obtaining a forward mutated α-1,2-fucosyltransferase mutant by microfluidic sorting of droplets.

A first object of the present invention is to provide a high-throughput method for screening α-1,2-fucosyltransferase, which includes the following steps:
Step S1: constructing an indicator strain of 2′-fucosyllactose, where the indicator strain includes a first expression vector and a second expression vector, the first expression vector includes an α-L-fucosidase coding gene, the second expression vector includes a lac operon and a fluorescent reporter gene, and α-L-fucosidase degrades 2′-fucosyllactose into lactose, and regulates, together with the lac operon, the expression of the fluorescent reporter gene, so that the content of 2′-fucosyllactose is correlated with the fluorescence intensity;
Step S2: constructing a chassis strain, where the chassis strain expresses an L-fucokinase coding gene and an α-1,2-fucosyltransferase mutant library;
Step S3: embedding the chassis strain in single-cell droplets and culturing the droplets, to obtain a droplet microfluidic chip, and culturing the droplets in a medium containing fucose and lactose; and
Step S4: subjecting the cell suspension of the indicator strain to one-to-one fusion with the droplets, co-culturing, microfluidically sorting the droplets according to the fluorescence intensity in the fused droplets, to obtain a chassis strain comprising a forward mutant of α-1,2-fucosyltransferase.

Further, in Step S1, the first expression vector further includes a signal peptide MalE coding gene.

Further, in Step S2, the chassis strain further includes a temperature-controlled expression vector, and the temperature-controlled expression vector regulates the expression of a β-galactosidase coding gene.

Further, in Step S3, the droplets are cultured at a first temperature, to enable the chassis strain to covert fucose and lactose into 2′-fucosyllactose, and then the droplets are cultured at a second temperature until the chassis strain completely degrades lactose.

Further, the temperature-controlled expression vector is pBV220.

Further, in Step S2, the UDP-glucose-1-phosphate transferase gene wcaJ is knocked out from the chassis strain.

Further, in Step S3, the droplets are water-in-oil droplets.

Further, the oil phase is 2% Pico-Surf in Novec 7500.

Further, in Step S4, the absorbance of the cell suspension of the indicator strain at 600 nm is 10-12.

In an embodiment of the present invention, the absorbance of the cell suspension of the indicator strain at 600 nm is 10.

A second object of the present invention is to provide an α-1,2-fucosyltransferase mutant, which includes any one of the following modifications to a starting sequence having an amino acid sequence as shown in SEQ ID NO. 8:
(1) mutation of valine at position 93 to isoleucine;
(2) mutation of glutamic acid at position 50 to glycine;
(3) mutation of lysine at position 81 to methionine, and mutation of valine at position 173 to isoleucine; and
(4) mutation of lysine at position 102 to glutamic acid, and mutation of tryptophan at position 284 to cysteine.

A third object of the present invention is to provide a gene encoding the α-1,2-fucosyltransferase mutant.

A fourth object of the present invention is to provide an expression vector comprising the gene.

A fifth object of the present invention is to provide a host cell comprising the α-1,2-fucosyltransferase mutant, the gene or the expression vector1.

Further, the host cell is bacterial or fungal cells.

Further, the host cell is E. coli cells.

In an embodiment of the present invention, the host cell is E. coli K12 MG1655 cells.

A sixth object of the present invention is to provide use of the α-1,2-fucosyltransferase mutant, the gene, the expression vector, or the host cell in the production of 2′-fucosyllactose.

A seventh object of the present invention is to provide an E. coli strain producing 2′-fucosyllactose. In the E. coli strain, the β-galactosidase coding gene and the UDP-glucose-1-phosphate transferase gene coding gene are knocked out, and the L-fucokinase coding gene and the α-1,2-fucosyltransferase mutant according to claim 9 are heterogeneously expressed.

An eighth object of the present invention is to provide a microbial agent containing the E. coli strain.

A ninth object of the present invention is to provide a method for producing 2′-fucosyllactose, which includes the step of production by fermentation with the E. coli strain according to claim 14 or the microbial agent according to claim 15.

### Beneficial Effects of the Present Invention

The present invention provides a high-throughput method for screening an α-1,2-fucosyltransferase mutant. By constructing an indicator strain of 2′-fucosyllactose and a chassis strain, the quick screening of the α-1,2-fucosyltransferase mutant is realized by microfluidic screening of droplets, thus improving the screening efficiency. With the aid of the positive correlation between the fluorescence intensity of the fluorescent reporter gene and the content of 2′-fucosyllactose, the activity of α-1,2-fucosyltransferase can be visually and quickly evaluated, to screen out a chassis strain highly producing 2′-fucosyllactose. By a temperature-controlled expression vector, the transcription of a β-galactosidase coding gene can be controlled at various temperature, to solve the possible noise interference caused by the substrate lactose, and further optimize the screening process. The enzyme activity of the mutant FutC^{V93I} screened by the method provided in the present invention is 2.31 times that of FutC, thus improving the screening efficiency of α-1,2-fucosyltransferase and the production efficiency of 2′-fucosyllactose, The present invention has a good industrial application prospect, and is beneficial to the industrial production, large-scale application and popularization of 2′-fucosyllactose in the fields of food, medicine, and others.

### BRIEF DESCRIPTION OF THE DRAWINGS

To make the disclosure of the present invention more comprehensible, the present invention will be further described in detail by way of specific embodiments of the present invention with reference to the accompanying drawings, in which:

FIG. 1 shows a map of plasmid pJ23110-MalE-AFCA in Example 1 of the present invention;

FIG. 2 shows a map of plasmid pACYC-T7lac-sfGFP in Example 1 of the present invention;

FIG. 3 shows a fluorescence response curve of indicator strain BL01 of 2′-fucosyllactose in Example 1 of the present invention;

FIG. 4 schematically shows a salvage synthesis pathway of 2′-fucosyllactose;

FIG. 5 shows a map of plasmid pRSF-trc-futC-fkp in Example 2 of the present invention;

FIG. 6 shows the fermentation verification of an α-1, 2-fucosyltransferase screening chassis strain in Example 2 of the present invention;

FIG. 7 schematically shows a screening process with a high-throughput screening platform for α-1, 2-fucosyltransferase in Example 3 of the present invention;

FIG. 8 shows a map of plasmid pET28a-gst-futc for protein expression in Example 4 of the present invention;

FIG. 9 shows the result of gel electrophoresis of protein FutC and a mutant thereof in Example 4 of the present invention; and

FIG. 10 shows the detection results of enzyme activity and enzyme level of α-1, 2-fucosyltransferase and a mutant thereof in Example 4 of the present invention.

### DETAILED DESCRIPTION OF THE PREFERRED EMBODIMENTS

The present invention will be further described below with reference to the accompanying drawings and specific examples, so that those skilled in the art can better understand and implement the present invention; however, the present invention is not limited thereto.

The temperature-controlled plasmid pBV220 used in the examples is disclosed in the literature: Mutation of λpL/pR-cI⁸⁵⁷ system for production of bacterial ghost in Escherichia coli. [J]. Chinese Journal of Biotechnology, 2012, 28(12): 1423-1430. The culture media and stock solutions involved in the examples are as follows: LB liquid medium: yeast extract 5 g/L; peptone 10 g/L, and sodium chloride 10 g/L; TB solid medium: peptone 12 g/L, yeast extract 24 g/L; glycerol 4 mL/L, potassium dihydrogen phosphate 2.31 g/L, and dipotassium hydrogen phosphate trihydrate 16.43 g/L;
fucose stock solution: fucose 150 g/L; and
lactose stock solution: lactose 200 g/L.

The detection methods of α-1,2-fucosyltransferase activity and 2′-fucosyllactose involved in the examples are as follows:
(1) The activity of wild-type FutC and a mutant thereof are determined by measuring the titer of 2′-FL. 300 L of a reaction mixture (containing 50 mM acetic acid buffer pH=6.0, 2 mM GDP-L-fucose, 5 mM lactose, 2 mM divalent magnesium ion and 200 g pure enzyme) is incubated at 37° C. for 20 min. The reaction is terminated by boiling the reaction solution for 10 min. Under the standard experimental conditions, the unit of enzyme activity is defined as the amount of enzyme needed to produce 1 mol 2′-FL per minute.
(2) 1 mL of a fermentation liquid or the above reaction solution is centrifuged at 10,000 rpm for 10 min. The supernatant is collected, and the 2′-FL content is determined by HPLC.

HPLC detection conditions: High performance liquid chromatography (HPLC) system (Waters e2695); Chromatographic column: Rezex ROA-Organic acid H⁺ (8%); Detector: Waters 2414RIDetector differential detector; Mobile phase: 5 mM H₂SO₄; Flow rate: 0.6 mL/min; Column temperature: 60° C.; Volume of injection: 10 μL.

The strains involved in the examples are as follows:

TABLE 1
Strains and genotypes

| Strain name | Genotype |
|---|---|
| E. coli BL21(DE3) | F⁻, ompT, hsdSB (rB-mB-), gal, dcm(DE3) |
| E. coli K12 | F-λ- ilvG- rfb-50 rph-1 |
| MG1655 | |
| E. coli JM109 | recA1, endA1, thi, gyrA96, supE44, |
| | hsdR17Δ(lac-proAB)/F′[traD36, proAB⁺, |
| | lacIq, lacZΔM15] |
| BL01 | E. coli BL21(DE3), pJ23110-MalE-AFCA, |
| | pACYC-T7lac-sfGFP |
| MGZ1 | E. coli MG1655ΔlacZΔwcaJ |
| MGZ2 | E. coli MG1655 ΔlacZΔwcaJ |
| | pRSF-trc-futC-fkp, pbv220-TB-lacZ |
| BLM0 | E. coli BL21(DE3), pET28a-gst-futc |
| BLM1 | E. coli BL21(DE3), pET28a-gst-futc^{E50G} |
| BLM2 | E. coli BL21(DE3), pET28a-gst-futc^{K102E/W284C} |
| BLM3 | E. coli BL21(DE3), pET28a-gst-futc^{K81M/V173I} |
| BLM4 | E. coli BL21(DE3), pET28a-gst-futc^{V93I} |

TABLE 2
Primers and sequences

| Primer | |
|---|---|
| name | Sequence (5′-3′) |
| AfcA-F | AATCTACTGGTATTGTACGTATGGTTATTGCATCAGTTGAAGATG |
| | (SEQ ID NO. 10) |
| AfcA-R | TGTGTTGAAATATGTTTTGTTGATGCTTTTTTTGTAATTGTATATGT |
| | TGT (SEQ ID NO. 11) |
| PJ23110-F | ACGTACAATACCAGTAGATTTCATAAACATTTTTTTTCCTCCTTTT |
| | CTC (SEQ ID NO. 12) |
| PJ23110-R | ACAAAACATATTTCAACACAATACAAATGGGTTAGTTAAAAAAGC |
| | AG (SEQ ID NO. 13) |
| male-F | CGCATTATCCGCATTAACGACGATGATGTTTTCCGCCTCGGCTCTC |
| | GCCATGGTTATTGCATCAGTTGAAGAT (SEQ ID NO. 14) |
| male-R | GTCGTTAATGCGGATAATGCGAGGATGCGTGCACCTGTTTTTATTT |
| | TCATGGTATATCTCCTTGTGTGAGTTAATCT (SEQ ID NO. 15) |
| GFP-F | TAAGGAGATATAATGAGCAAAGGAGAAGAACTTTTCA |
| | (SEQ ID NO. 16) |
| GFP-R | GGTGGCAGCAGTTATTTGTAGAGCTCATCCATGCCA |
| | (SEQ ID NO. 17) |
| PACY-F | TACAAATAACTGCTGCCACCGCTGAGC (SEQ ID NO. 18) |
| PACY-R | TCCTTTGCTCATTATATCTCCTTATTAAAGTTAAACAAAATTATTTC |
| | TACAG (SEQ ID NO. 19) |
| fkp-F | CAACGCCTAAtttgtttaactttaagaaggagatataccatgCAAAAG (SEQ ID NO. |
| | 20) |
| fkp-R | accagatcaatacaaataataaacgcatgagAAAGCCCC (SEQ ID NO. 21) |
| futc-F | TGAGTGAGCTAACTTACTTGACAATTAATCATCCGGCTCGT (SEQ |
| | ID NO. 22) |
| futc-R | gttaaacaaaTTAGGCGTTGTACTTCTGGCTCTTGACCTCGAAG (SEQ |
| | ID NO. 23) |
| RSF-F | tgtattgatctggtACACGGTCACACTGCTTCC (SEQ ID NO. 24) |
| RSF-R | GTAAGTTAGCTCACTCATTAGGCACCGGGATCTCGACCGATGCCC |
| | TT (SEQ ID NO. 25) |
| pbv220-F | GGCTGTTTTGGCGGATGAGAGAAGATTTTCAGCCTGATACAGATT |
| | AAATC (SEQ ID NO. 26) |
| pbv220-R | gctgtttcctgtgtgaCCTCCTTAATTTTTAACCAATGCTTCGTTTCGTATC |
| | ACACA (SEQ ID NO. 27) |
| lacz-F | GGTTAAAAATTAAGGAGGtcacacaggaaacagctatgaccatgattacggattcact |
| | (SEQ ID NO. 28) |
| lacz-R | CTCTCATCCGCCAAAACAGCCAAttatttttgacaccagaccaactg (SEQ ID |
| | NO. 29) |
| pet28a-f | CCATCATCACCACTAAGATCCGGCTGCTAACAAAGCCCG (SEQ ID |
| | NO. 30) |
| pet28a-r | GGTATATCTCCTTCTTAAAGTTAAACAAAATTATTTCTAGAGGGGA |
| | ATTGT (SEQ ID NO. 31) |
| gst-f | CTTTAAGAAGGAGATATACCATGTCCCCTATACTAGGTTATTGG |
| | (SEQ ID NO. 32) |
| gst-r | CACGTTTGGTGGTGGCGACCATCCTCCAAAAatgGCC (SEQ ID NO. |
| | 33) |
| gst-futc-f | CGACCATCCTCCAAAAatgGCCTTTAAAGTCGTCCAAATC (SEQ ID |
| | NO. 34) |
| gst-futc-r | GGATCTTAGTGGTGATGATGGTGATGGGCGTTGTACTTCTGGCTC |
| | TTGACC (SEQ ID NO. 35) |

### Example 1: Indicator Strain of 2′-fucosyllactose

(1) Production of Plasmid pJ23110-MalE-AFCA

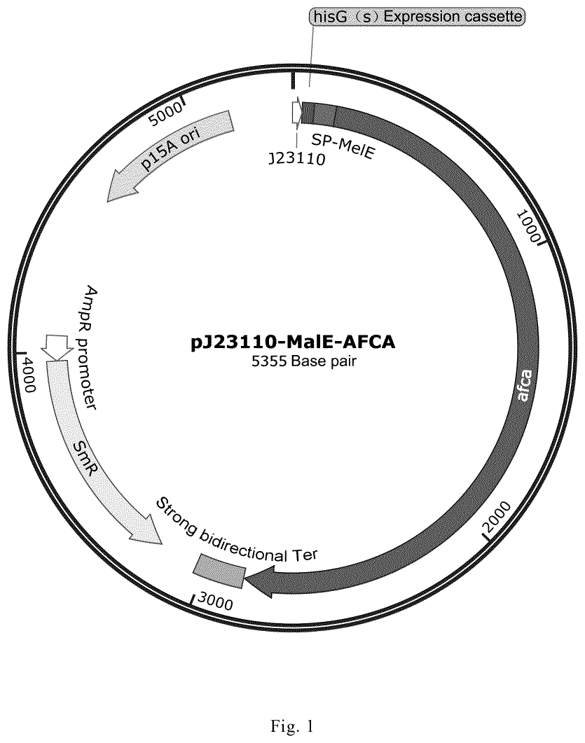

A constitutive promoter PJ23110 (the nucleotide sequence was as shown in SEQ ID NO. 1) was used for the expression of a signal peptide MalE (the amino acid sequence was as shown in SEQ ID NO. 2) and a protein AfcA coding gene (the nucleotide sequence was as shown in SEQ ID NO. 3). The α-L-fucosidase AfcA could specifically degrade 2′-fucosyllactose to obtain lactose, and the signal peptide MalE could efficiently transport the protein AfcA to the periplasmic space. The specific steps were as follows. The gene sequence of afcA from Bifidobacterium bifidum was searched from NCBI, and the DNA sequence after codon optimization was sent to Suzhou GENEWIZ, Inc. for synthesis. Then the synthetic gene afcA and plasmid p15A-PJ23110 were amplified by PCR using primers AfcA-F/AfcA-R and PJ23110-F/PJ23110-R respectively. The PCR products were ligated by using a seamless cloning kit, and the obtained plasmid was named pJ23110-AFCA. The plasmid pJ23110-AFCA was subjected to whole plasmid PCR using primers male-F/male-R having homolog arms. The PCR product was purified and transformed into E. coli JM109 competent cells. The positive clone confirmed to be correct was pJ23110-MalE-AFCA (having a map as shown in FIG. 1). The plasmid was extracted by using a plasmid extraction kit.

(2) Production of Plasmid pACYC-T7Lac-sfGFP

With plasmid pACYCDuet-1 as a backbone, a fluorescent reporter gene sfgfp (the nucleotide sequence was as shown in SEQ ID NO. 5) was expressed by using an inducible promoter P_{T7lac} (the nucleotide sequence was as shown in SEQ ID NO. 4). The specific steps were as follows.

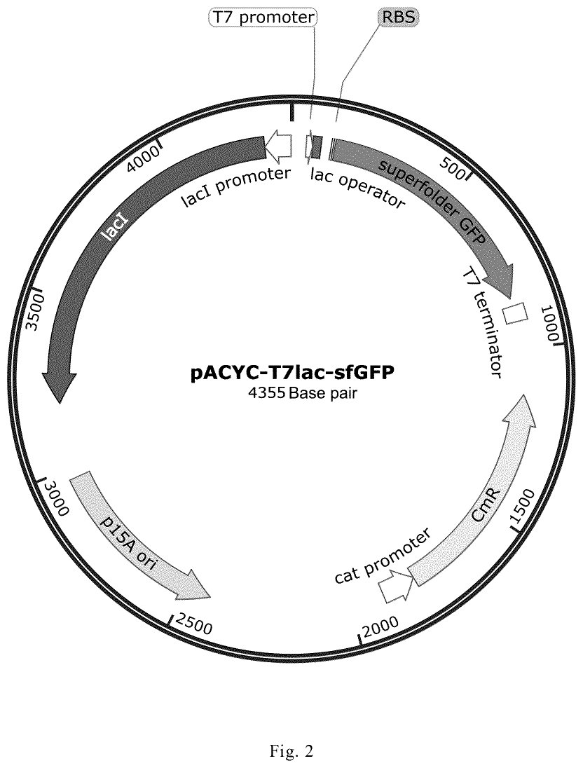

The green fluorescent protein gene sfgfp and pACYCDuet-1 were amplified by PCR using primers GFP-F/GFP-R and PACY-F/PACY-R. The PCR products were ligated by using a seamless cloning kit, to obtain plasmid pACYC-T7lac-sfGFP (having a map as shown in FIG. 2). The plasmid was transformed into E. coli JM109 competent cells. The positive clone confirmed to be correct was pACYC-T7lac-sfGFP. The plasmid was extracted by using a plasmid extraction kit.

(3) Construction and Identification of Indicator Strain of 2′-fucosyllactose

The plasmids pJ23110-MalE-AFCA and pACYC-T7lac-sfGFP constructed in Steps (1) and (2) were co-transformed into E. coli BL21(DE3) chemically competent cells. The cells were plated into a corresponding LB medium with two antibodies (streptomycin and chloramphenicol resistance). The normally grown transformant was indicator strain BL01 of 2′-fucosyllactose. The lacI gene provided in the plasmid pACYC-T7lac-sfGFP expresses the LacI repressor protein, which binds to lacO fragment to inhibit the transcription of the fluorescent reporter gene sfgfp. When the LacI repressor protein is bound to lactose obtained by degradation of 2′-fucosyllactose by the α-L-fucosidase AfcA provided by the plasmid pJ23110-MalE-AFCA, the transcription inhibition of the fluorescent reporter gene will be eliminated. Therefore, the indicator strain can be used to sense the concentration of 2′-fucosyllactose to linearly regulate the transcription intensity of the fluorescent reporter gene.

The LB liquid medium was added to a 96-well microplate in an amount of 200 μL per well. Then 2 μL of a seed culture containing the 2′-fucosyllactose indicator strain BL01 was inoculated, and continuously cultured on a constant-temperature shaker for 96-well microplates at 37° C. and 900 rpm until OD₆₀₀=0.6. Various concentration gradients of 2′-FL (0, 62.5, 125, 250, 500, 1000, 2000, and 4000 mg/L), were added. The cells were continuously cultured on the shaker for another 6 h, and the cell density and fluorescence intensity were detected. The fluorescence intensity of the culture was read on a microplate reader, where the gain was set to 60, the excitation wavelength was 480 nm, and the emission wavelength was 530 nm. The OD₆₀₀ value was determined. The fluorescence level of single cells was characterized by fluorescence intensity/OD₆₀₀.

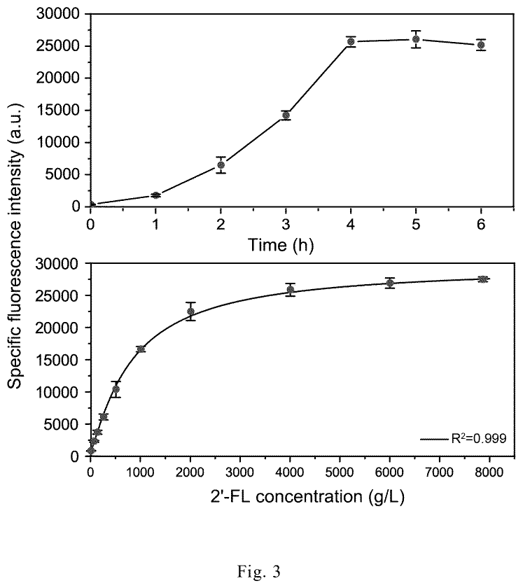

The fluorescence response curve of the 2′-fucosyllactose indicator strain BL01 is shown in FIG. 3. When the concentration of 2′-FL is 62.5-4000 mg/L, the outputted fluorescence signal value is positively correlated with the concentration of 2′-FL. When the concentration of 2′-FL is 62.5 mg/L, the Fl/D₆₀₀ value of the sensor is 687±17. When the concentration of 2′-FL is 4000 mg/L, the Fl/D₆₀₀ value of the sensor is 69457±4000, which is 101.02 times the value when the concentration is 62.5 mg/L. A logistic model was used for non-linear fitting of the data, to obtain a fitted equation: y=13885-13376/(1+(x/530)1.18)(R²=0.999). Therefore, the working range of the 2′-fucosyllactose indicator strain is 62.5-4000 mg/L.

Then, to verify the accuracy and reliability of the indicator strain, 6 2′-FL samples with different concentrations were prepared, and analyzed and compared by using the indicator strain and HPLC respectively. The results are shown in Table 3. The corresponding 2′-FL concentration was obtained by converting the obtained fluorescence ratio with the above fitted equation, and the error between the 2′-FL concentration and the standard value (HPLC) was less than 5%, indicating that the 2′-FL indicator strain constructed in this example had a high reliability.

TABLE 3
Accuracy evaluation of 2′-FL indicator strain

| | | Specific | | | |
|---|---|---|---|---|---|
| | | fluorescence | Measure- | Standard | |
| | | intensity | ment | value | Error |
| No. | Fitted equation | (a.u.) | (mg/L) | (mg/L) | (%) |
| 1 | y = 13885 − | 2970 ± 124 | 150 | 143 | 5 |
| 2 | 13376/(1 + | 8318 ± 325 | 706 | 685 | 2 |
| 3 | (x/530)1.18) | 9662 ± 527 | 1021 | 1063 | 4 |
| 4 | R² = 0.999 | 11561 ± 498 | 1987 | 2088 | 5 |
| 5 | | 12072 ± 788 | 2548 | 2498 | 2 |
| 6 | | 12724 ± 657 | 3894 | 3804 | 2 |

### Example 2: Construction of α-1, 2-fucosyltransferase Screening Chassis Strain

(1) Production of E. coli MG1655ΔlacZΔwcaJ

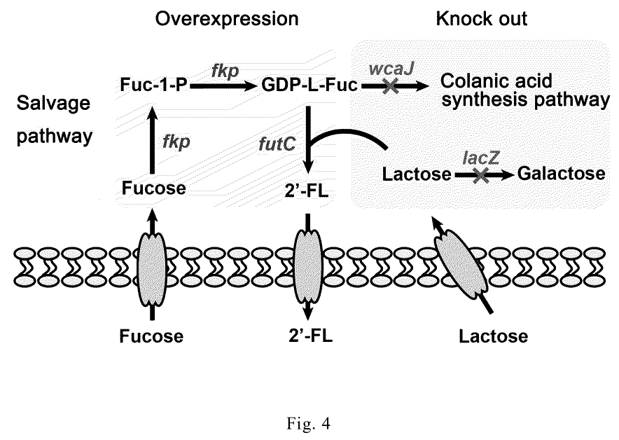

E. coli K12 MG1655 was used as a starting strain. The β-galactosidase gene lacZ (the nucleotide sequence was as shown in Gene ID: 945006) and the UDP-glucose-1-phosphate transferase gene wcaJ (the nucleotide sequence was as shown in Gene ID: 946583) in the starting strain were knocked out by a method as described in Patent Publication No. CN110804577A entitled Method for constructing recombinant strain efficiently producing 2′-fucosyllactose and use thereof. A recombinant strain E. coli MG1655ΔlacZΔwcaJ was obtained, which was designated as MGZ1. lacZ and wcaJ are both a gene in an alternative pathway to the salvage synthesis pathway of 2′-fucosyllactose. Knocking out these two genes can increase the utilization rate of lactose and GDP-L-fucose in the 2′-FL metabolic pathway, and further guide the metabolic flow to the target metabolite, thus further increasing the production of 2′-FL. The process of the salvage synthesis pathway of the 2′-fucosyllactose is shown in FIG. 4.

(2) Production of Plasmid pRSF-Trc-futC-Fkp

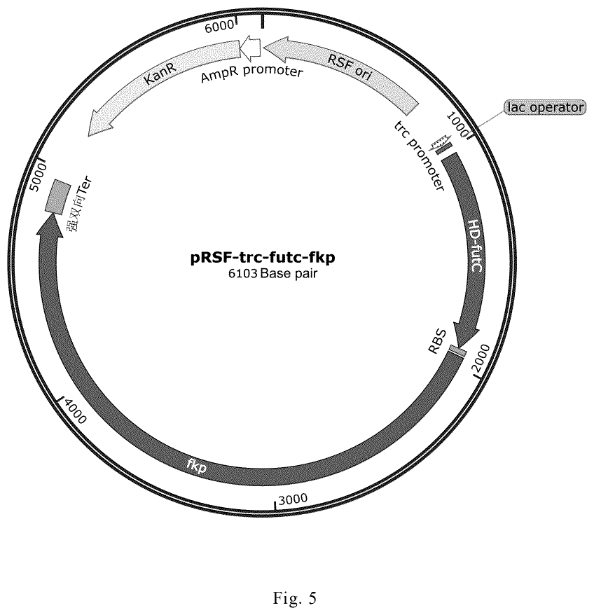

Using the genome of E. coli as a template, the fkp gene (the nucleotide sequence was as shown in SEQ ID NO. 6) was obtained by PCR amplification using primers fkp-F/fkp-R, which was used to express L-fucokinase FKP that could convert fucose into GDP-L-fucose. The plasmid pRSFDuet-1 was amplified by PCR using primers RSF-F/PSF-R. The amino acid sequence of FutC from Helicobacter pylori (ATCC NO. 26695) was searched from NCBI, and the DNA sequence after codon optimization was sent to Suzhou GENEWIZ, Inc. for synthesis. By using primers FutC-F/FutC-R and with FutC (the nucleotide sequence was as shown in SEQ ID NO. 7, the amino acid sequence was as shown in SEQ ID NO. 8, and it was used for expressing α-1,2-fucosyltransferase to convert GDP-L-fucose and lactose into 2′-fucosyllactose) as a template, FutC was mutated by using the QuickMutation™ Random Mutagenesis Kit manufactured by Beyotime Biotechnology to construct a mutant library. The PCR products were ligated by using a seamless cloning kit, and transformed into E. coli JM109 competent cells after purification. The positive clone confirmed to be correct was pRSF-trc-futC-fkp (having a map as shown in FIG. 5). The plasmid was extracted by using a plasmid extraction kit. The obtained plasmid was a mutant library plasmid.

### (3) Production of Plasmid Pbv220-TB-lacZ

Using the genome of E. coli as a template, the PCR amplification was performed using primers lacZ-F/lacZ-R, to obtain the lacZ gene (the nucleotide sequence was as shown in Gene ID: 945006). The temperature controlled plasmid pbv220 was amplified by PCR using primers pbv220-F/pbv220-R. The PCR products were ligated by using a seamless cloning kit, and transformed into E. coli JM109 competent cells after purification. The positive clone confirmed to be correct was pbv220-TB-lacZ. The plasmid was extracted by using a plasmid extraction kit. In the plasmid, the CI⁸⁵⁷ gene (the nucleotide sequence was as shown in SEQ ID NO. 9) regulated to express by the promoter P_{L} and the lacZ gene regulated to express by the promoter P_{R}, enabled the repressor protein expressed by the CI⁸⁵⁷ gene to bind with the promoter P_{R} at 37° C. to inhibit the transcription of the lacZ gene, and eliminated the transcription inhibition on the lacZ gene at 42° C.

(4) Confirmation of α-1, 2-fucosyltransferase Screening Chassis Strain

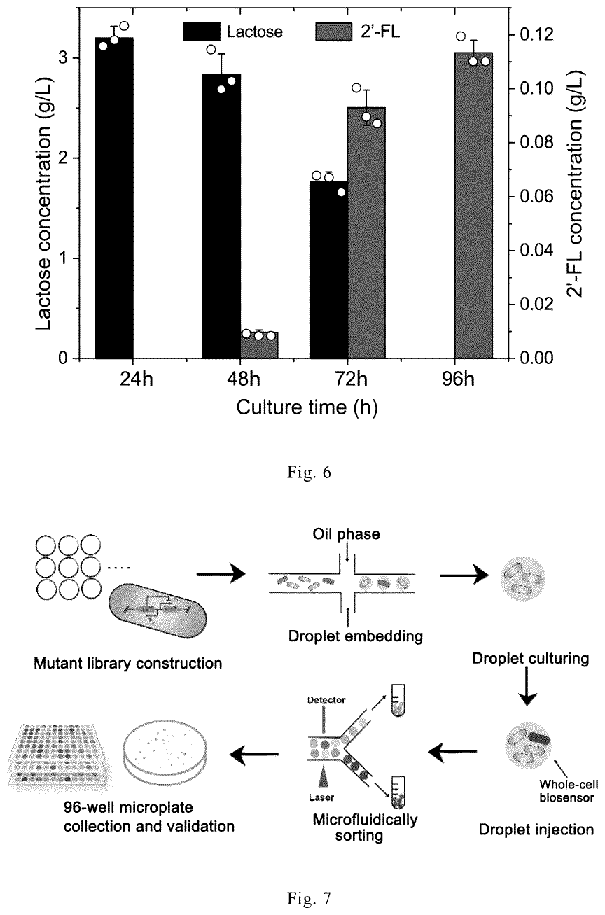

The plasmids obtained in Steps (2) and (3) were co-transformed into E. coli MG1655ΔlacZΔwcaJ chemically competent cells, to obtain the engineered strain MGZ2. MGZ2 with original protein was inoculated into the LB liquid medium, and cultured overnight at 37° C. and 200 rpm for 12 h, to obtain a seed culture. 500 μL of the seed culture was inoculated into 25 mL of TB medium, cultured at 37° C. and 200 rpm until OD₆₀₀ was 0.6-0.8. IPTG at a final concentration 0.3 mM was added, and lactose and fucose respectively at a final concentration of 2 g/L and 3 g/L were added. The cells were further inducibly incubated at 37° C. and 200 rpm for 72 h. The incubation temperature was adjusted to 42° C. and the cells were further cultured for 12 h. 1 mL of the fermentation liquid was centrifuged at 10,000 rpm for 10 min. The supernatant was collected, and the yields of 2′-fucosyllactose and lactose were determined by HPLC. The results are shown in FIG. 6. The final concentration of 2′-fucosyllactose is 0.11 g/L and the final concentration of lactose is 0, indicating that MGZ2 can not only inhibit the transcription of the lacZ gene at 37° C. to produce 2′-fucosyllactose, but also release the transcription inhibition on the lacZ at 42° C. to completely degrade lactose. Therefore, the possible noise interference caused by the substrate lactose is solved, and all lactose in the screening process is ensured to be obtained by degrading 2′-fucosyllactose with the AfcA protein in the indicator strain, to meet the screening requirement.

### Example 3: Droplet Microfluidic Screening for Construction of Recombinant Strain

### (1) Optimization of Droplet Generation Conditions

The α-1, 2-fucosyltransferase screening chassis strain MGZ2 was washed three times with the LB medium and resuspended. The OD₆₀₀ value was adjusted to 0.05, Single cell droplets were prepared by using a droplet generation chip. The aqueous phase was TB medium as a suspension containing MGZ2 cells. The oil phase was 2% Pico-Surf in FC40, 2% Pico-Surf in Novec 7500 or Bio-Rad droplet generation oil. The oil phase and the aqueous phase were respectively drawn into a 1 mL sterile syringe, which was installed in a micro-injection pump and connected to a chip with a conduit. The flow rate of the oil phase was set at 5 L/min, and the flow rate of the aqueous phase was set at 2 μL/min. Single-cell droplets with a diameter of about 30 m were generated. The droplets were collected with a 1 mL sterile syringe. The syringe filled with single cell droplets was cultured in an incubator at 37° C., and the droplets were sampled at various times to evaluate the stability of the droplets. The oil phase of the most stable droplets was used for subsequent experiments.

The oil phase of the most stable droplets in the above experiment was 2% Pico-Surf in Novec 7500. According to the above conditions, the droplets of the MGZ2 strain were generated (the resuspension medium was TB medium, and final concentrations of 0.3 mM IPTG, 3 g/L lactose, and 2 g/L fucose were added). The syringe filled with single cell droplets was respectively placed an incubator at 37° C. for 72 h and at 42° C. for 12 h sequentially, to eliminate the interference of the substrate lactose. Then, the subsequent experiments were carried out.

### (2) Optimization of Droplet Injection Conditions

Sterile water was used as the aqueous phase and 2% Pico-Surf in Novec 7500 was used as the oil phase, to generate water-in-oil single emulsion droplets. The droplets were collected with a 1 mL sterile syringe. and re-injected into the microfluidic chip. In the presence of the electric field force, the cell suspension in the sensor was one-to-one fused with the passing droplets at a speed of 0.8 μL/min. The fused droplets were collected and cultured in a droplet microfluidic incubator at 37° C. After 24 h, the sample was taken out, and the injection of the strain in the droplets was observed under a bright field and a dark field of a fluorescence microscope. The concentration of the reporter strain with stable droplet size and green fluorescence in each droplet was used as the optimum concentration, and the OD₆₀₀ of the indicator strain was 10 in this case.

### (3) Droplet Screening

The collected single cell droplets were cultured in an incubator at 37° C., where the culture medium was TB medium. The samples were taken and the fluorescence changes in the droplets were observed at various times. Droplet microfluidic screening was carried out at an appropriate culture time (12 h). During screening, the syringe filled with the oil phase and the droplets was installed in a micro-injection pump, and the outlet of the syringe was connected to a corresponding inlet of a microfluidic droplet sorting chip by a conduit. The flow rate of the oil phase was set at 10 L/min, and the flow rate of the droplets was set at 0.5 L/min. In this case, the droplet flow rate was about 500-1000 droplets per second. The blue laser (488 nm) was aimed at a detection spot on the screening chip, and the green fluorescence signal (520 nm) of each droplet was collected. The droplets with the highest fluorescence signal of about 30% was energized at a voltage set to 300 V or 500 V, so that the target droplets flowed to a collection outlet of the screening chip under the electrostatic action, and the rest droplets flowed out from the waste liquid outlet. The collected droplets were coated on an LB plate. The screening process was shown in FIG. 3. The screened strains were sequenced to obtain mutants FutC^{V93I}, FutC^{K81M/V173I}, FutC_{E50G} and FutC^{K102E/W284C}.

### Example 4: Example 4: Determination of Enzyme Activity

### (1) Expression of Recombinant Protein

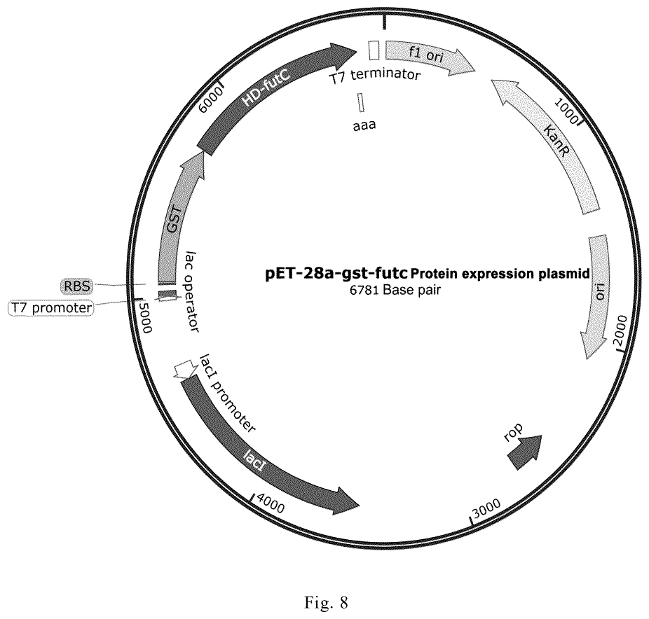

The recombinant wild-type FutC and its mutant proteins obtained in Example 3 were overexpressed in E. coli BL21(DE3) respectively. The GST protein tag was amplified by PCR using primers gst-f/gst-r. The recombinant wild-type FutC and its mutants were amplified by PCR using primers gst-futc-f/gst-futc-r. The plasmid vector pET28a was amplified by PCR using primers pet28a-f/pet28a-r. The PCR products were ligated by using a seamless cloning kit, to obtain plasmid pET28a-gst-futc (having a map as shown in FIG. 8). The plasmid was transformed into E. coli JM109 competent cells. The positive clone confirmed to be correct was pET28a-gst-futc. The plasmid was extracted by using a plasmid extraction kit. The plasmid pET28a-gst-futc constructed was transformed into E. coli BL21(DE3) chemically competent cells. The cells were plated into an LB medium, and the normally growing transformant was futC overexpressing strain BLM0-BLM4.

The cells were shaken (220 r/min) overnight in the LB medium at 37° C. 4 ml of fresh cells grown in the LB medium were transferred to fresh TB medium (200 mL) in a 500 mL unbaffled flask, incubated until the OD₆₀₀ was 0.6. IPTG at a final concentration of 0.3 mM was added to the culture medium to induce the expression of the target protein. After 20 h incubation at 20° C., the recombinant cells were collected by centrifugation at 8000 g for 10 min.

### (2) Protein Purification and Concentration Detection

The recombinant FutC protein and its mutants were separated and purified by Ni²⁺ affinity chromatography. The cell precipitate was resuspended in the lysis buffer, and ultrasonically homogenized on the ice for 20 min with alternation of an on-time of 1 s and an off-time of 2 s. The homogenized mixture was centrifuged at 8000 g for 10 min, and filtered through a 0.45 μm filter to obtain a crude enzyme solution. The collected supernatant was loaded onto GST agarose purification resin (Sangon Bio, Shanghai). 2 columns of protein purification buffer A (20 mM Tris-HCl buffer, pH=8.0, adjusted to pH 6.5 with concentrated hydrochloric acid, then made up to a volume of 2 L with ultrapure water, and then filtered through a 0.45 μM filter) was used to balance the column. Then, 10% protein purification buffer B (500 mM imidazole, 20 mM Tris-HCl buffer, pH=8.0, adjusted to pH 6.5 with concentrated hydrochloric acid, then made up to a volume of 2 L with ultrapure water, and then filtered through a 0.45 M filter) was used to elute the unbound protein. Finally, the target protein was eluted with 100% protein purification buffer B pH 6.5. The concentration of the purified enzyme was determined by a BCA protein assay kit (Beyotime, Shanghai, China). The results are shown in Table 4.

TABLE 4
Protein concentrations corresponding to the enzyme and its mutants

| | Protein |
|---|---|
| Enzyme and | concentrations |
| its mutants | (mg/mL) |
| FutC | 0.55 |
| FutCV93I | 0.59 |
| FutCK81M/V173I | 0.61 |
| FutCE50G | 0.55 |
| FutCK102E/W284C | 0.54 |

### (3) Analysis by SDS-PAGE

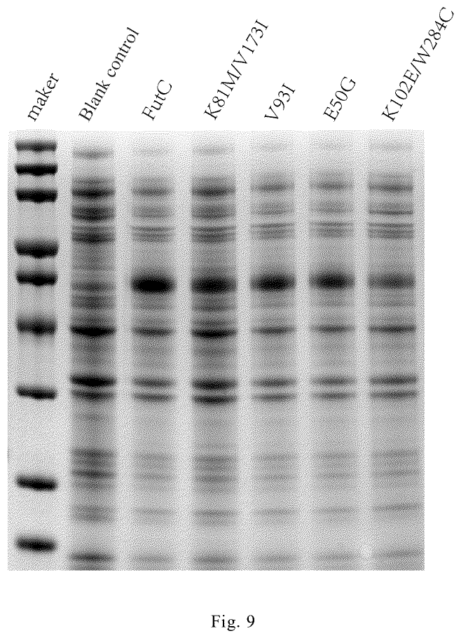

Obtaining and processing operations of protein samples: The cell wall of the bacteria was ruptured. 1-2 mL of the obtained solution was centrifuged at 4000 g and 4° C. for 4 min. The supernatant was discarded. The cells were gently pipetted with a lysis buffer (20 mM Tris-HCl buffer pH 8.0, adjusted to pH 6.5 with concentrated hydrochloric acid, and then made up to 2 L with ultrapure water). The above operations were repeated twice. The washed sample was treated on MP Fastprep-24 5G Instrument for 40 s, and then centrifuged at 12000 rpm and 4° C. for 2 min. The supernatant was the protein sample. Sample treatment operations: The protein sample obtained above was mixed with NuPAGE™ 4×LDS sample buffer from Thermo Fisher Scientific, then denaturated in a metal bath at 98° C. for 10 min, and cooled to room temperature before use. Gel electrophoresis operation: The protein precast gel was fixed in a protein electrophoresis cell, a SDS-PAGE buffer was added to submerge the protein gel hole, and a proper amount of the treated protein sample and Protein Maker were aspirated into the protein gel hole. The voltage was at 120 V and the protein was subjected to electrophoresis for 120 min. Staining and destaining operation of protein gel: The protein gel was placed in a gel box with an appropriate amount of Coomassie brilliant blue staining solution, and stained for 30 min. After staining, the staining solution was poured back into the flask, and the protein gel was destained by adding water or a destaining solution. When the protein band was clearly visible, the protein gel was placed in a gel imaging system and observed. The result of protein gel electrophoresis is shown in FIG. 9.

### (4) Enzyme Activity Detection

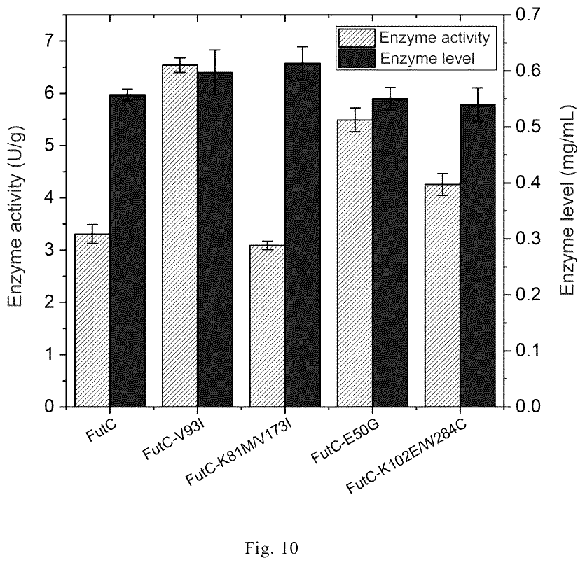

The activity of wild-type FutC and its mutants was determined by detecting the titer of 2′-FL. The reaction solution was 300 μL, including 20 mM Tris-HCl buffer (pH=6.5), 2 mM GDP-L-fucose, 5 mM lactose, 2 mM Mn²⁺ and 200 g pure enzyme. The reaction solution was incubated at 37° C. for 20 min and then boiled for 10 min to terminate the reaction. Under the standard experimental conditions, the unit of enzyme activity is defined as the amount of enzyme needed to produce 1 mol 2′-FL per minute. The concentrations of various carbohydrates (including lactose, GDP-L-fucose and 2′-fucosyllactose) in the reaction solution were analyzed by high performance liquid chromatography. The results are shown in FIG. 10.

Apparently, the above-described embodiments are merely examples provided for clarity of description, and are not intended to limit the implementations of the present invention. Other variations or changes can be made by those skilled in the art based on the above description. The embodiments are not exhaustive herein. Obvious variations or changes derived therefrom also fall within the protection scope of the present invention.

What is claimed is:

1. A high-throughput method for screening α-1,2-fucosyltransferase, comprising the steps of:
Step S1: constructing an indicator strain of 2′-fucosyllactose, wherein the indicator strain comprises a first expression vector and a second expression vector, the first expression vector comprises an α-L-fucosidase coding gene, the second expression vector comprises a lac operon and a fluorescent reporter gene, and α-L-fucosidase degrades 2′-fucosyllactose into lactose, and regulates, together with the lac operon, the expression of the fluorescent reporter gene, so that the content of 2′-fucosyllactose is correlated with the fluorescence intensity;
Step S2: constructing a chassis strain, wherein the chassis strain expresses an L-fucokinase coding gene and an α-1,2-fucosyltransferase mutant library;
Step S3: embedding the chassis strain in single-cell droplets and culturing the droplets, to obtain a droplet microfluidic chip, and culturing the droplets in a medium containing fucose and lactose; and
Step S4: subjecting the cell suspension of the indicator strain to one-to-one fusion with the droplets, co-culturing, microfluidically sorting the droplets according to the fluorescence intensity in the fused droplets, to obtain a chassis strain comprising a forward mutant of α-1,2-fucosyltransferase.

2. The high-throughput screening method according to claim 1, wherein in Step S1, the first expression vector further comprises a signal peptide MalE coding gene.

3. The high-throughput screening method according to claim 1, wherein in Step S2, the chassis strain further comprises a temperature-controlled expression vector, and the temperature-controlled expression vector regulates the expression of a β-galactosidase coding gene.

4. The high-throughput screening method according to claim 3, wherein in Step S3, the droplets are cultured at a first temperature, to enable the chassis strain to covert fucose and lactose into 2′-fucosyllactose, and then the droplets are cultured at a second temperature until the chassis strain completely degrades lactose.

5. The high-throughput screening method according to claim 3, wherein the temperature-controlled expression vector is pBV220.

6. The high-throughput screening method according to claim 1, wherein in Step S2, the UDP-glucose-1-phosphate transferase gene wcaJ is knocked out from the chassis strain.

7. The high-throughput screening method according to claim 1, wherein in Step S3, the droplets are water-in-oil droplets, and the oil phase is 2% Pico-Surf in Novec 7500.

8. The high-throughput screening method according to claim 1, wherein in Step S4, the absorbance of the cell suspension of the indicator strain at 600 nm is 10-12.

9. A α-1,2-fucosyltransferase mutant, having a sequence modified from a starting sequence as shown in SEQ ID NO. 8, by any of:
(1) mutation of valine at position 93 to isoleucine;
(2) mutation of glutamic acid at position 50 to glycine;
(3) mutation of lysine at position 81 to methionine, and mutation of valine at position 173 to isoleucine;
(4) mutation of lysine at position 102 to glutamic acid, and mutation of tryptophan at position 284 to cysteine.

10. A gene encoding the α-1,2-fucosyltransferase mutant according to claim 9.

11. An expression vector comprising the gene according to claim 10.

12. A host cell comprising the α-1,2-fucosyltransferase mutant according to claim 9.

13. Use of the host cell comprising the α-1,2-fucosyltransferase mutant according to claim 9 in the production of 2′-fucosyllactose.

14. An E. coli strain producing 2′-fucosyllactose, wherein in the E. coli strain, the β-galactosidase coding gene and the UDP-glucose-1-phosphate transferase coding gene are knocked out, and the L-fucokinase coding gene and the α-1,2-fucosyltransferase mutant according to claim 9 are heterogeneously expressed.

15. A microbial agent comprising the E. coli strain according to claim 14.

16. A method for producing 2′-fucosyllactose, comprising a step of production by fermentation with the E. coli strain according to claim 14.


SEQUENCE LISTING (35 sequences)

SEQ ID NO: 1 - DNA, 35 bp, unidentified
  source 1..35: mol_type = other DNA
tttacggctagctcagtcctaggtacaatgctagc

SEQ ID NO: 2 - protein, 26 aa, unidentified
MKIKTGARILALSALTTMMFSASALA

SEQ ID NO: 3 - DNA, 2696 bp, unidentified
  source 1..2696: mol_type = other DNA
atggttattgcatcagttgaagatggcggcgatggcgatacatcaaaagatgattggctgtggtataaacaaccggcatcacaaacagatgcaacagcaacagctggcggcaactatggcaatccggataataatagatggcaacaaacaacactgccgtttggcaatggcaaaattggcggcacagtttggggcgaagtttcaagagaaagagttacatttaatgaagaaacactgtggacgggcggcccgggctcatcaacatcatataatggcggcaataatgaaacaaaaggccaaaatggcgcaacactgagagcactgaataaacaactggcaaatggcgccgaaacagttaatccgggcaatctgacgggcggcgaaaatgcagcagaacaaggcaattatctgaattggggcgatatttatctggattatggctttaatgatacaacagttacagaatatagaagagatctgaatctgtcaaaaggcaaagcagatgttacatttaaacatgatggcgttacatatacaagagaatattttgcatcaaatccggataatgtcatggttgcaagactgacagcatcaaaagcgggcaaactgaattttaatgtttcaatgccgacaaatacaaattattcaaaaacgggcgagacgacaactgttaaaggcgatactctgacagttaaaggtgcactgggcaataatggcctgctgtataattcacaaattaaagttgttctggataatggcgaaggcacactgtcagaaggcagcgacggcgcatcattaaaggtttcagatgcaaaagcagttacactgtatattgcagcagcaacagattataaacaaaaatatccgtcatatagaaccggcgaaacagcggcggaagttaatacaagagttgcaaaagttgttcaagatgcagcaaataaaggctacacagcagttaaaaaagcacatattgatgatcattcagcaatttatgatagagttaaaattgatctgggccaaagcggccattcatcagacggtgcagttgcaacagatgcactgctgaaggcctatcaaagaggctcagcaacaacagcacaaaaaagagaactggaaacactggtttataaatatggcagatatctgacaattggctcatcaagagaaaattcacaactgccgtcaaatctgcaaggcatttggtcagttacagcgggcgataatgcacatggcaatacaccgtggggctcagattttcatatgaatgttaatctgcaaatgaattattggccgacatattcagcaaatatgggcgaactggcagaaccgctgattgaatatgttgaaggcctggttaaaccgggcagagttacagcaaaagtttatgcgggcgcagagactacaaatccggaaacaacaccgattggcgaaggcgaaggctatatggcacatacagaaaatacagcatatggctggacagcaccgggccaatcattttcatggggctggtcaccggcagcagttccgtggattctgcaaaatgtttatgaagcatatgaatatagcggcgatccggcactgctggatagagtttatgcattgctgaaggaggaatcacatttttatgttaattacatgttacataaagcgggctcatcaagcggcgatagactgacaacgggcgttgcatattcaccggaacaaggcccgctgggcacagatggtaatacatatgaatcatcactggtttggcaaatgctgaatgatgcaattgaagcagcaaaagcaaaaggcgatccggatggcctggttggcaatacaacagattgctcagcagataattgggcaaaaaatgatagcggcaattttacagatgcaaatgcaaatagatcatggtcatgcgcaaaatcactgctgaaaccgattgaagttggcgatagcggccaaattaaagaatggtattttgaaggtgcacttggcaagaagaaagatggctcaacaattagcggctatcaagcagataatcaacatagacatatgtcacatctgctgggcctgtttccgggcgatctgattacaattgataattcagaatatatggatgcagcaaaaacatcactgagatatagatgctttaaaggcaatgttctgcaaagcaatacgggctgggcaattggccaaagaattaattcatgggcaagaacgggcgatggcaatactacgtaccaactggttgaactgcaactgaaaaatgcaatgtatgcaaatctgtttgattatcatgcaccgtttcaaattgatggcaattttggcaatacaagcggcgttgatgaaatgctgctgcaatcaaactcaacatttacagatacagcgggcaaaaaatatgttaactatacaaatattctgccggcactgccggatgcatgggcgggcggctcagttagcggcctggttgcaagaggtaattttactgtaggcacaacatggaaaaatggcaaagcaacagaagttagactgacatcaaataaaggcaaacaagcagcagttaagattactgccggcggcgcacaaaattatgaagttaaaaatggcgacacagctgttaacgccaaagttgtcactaacgcagatggcgcatcacttctggtttttgatacaacagcgggcacaacatatacaattacaaaaaaagcatc

SEQ ID NO: 4 - DNA, 44 bp, unidentified
  source 1..44: mol_type = other DNA
ggaattgttatccgctcacaattcccctatagtgagtcgtatta

SEQ ID NO: 5 - DNA, 717 bp, unidentified
  source 1..717: mol_type = other DNA
atgagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaataa

SEQ ID NO: 6 - DNA, 2850 bp, unidentified
  source 1..2850: mol_type = other DNA
atgcaaaagttactgtctctcccatctaacttagtccagagctttcatgaattagaaagagtaaatcggactgactggttttgcacatccgacccggtcggaaaaaagcttggcagtggcggtggcacatcatggctgctcgaggaatgctataatgaatactctgacggcgcaactttcggagagtggctcgaaaaagagaaaagaatactgctccacgccggtgggcagtcccgccgtttgccgggttacgctcctagtggtaagatccttacgccggtcccagtatttcgctgggaaagaggacagcacctgggccagaaccttttgagtttacagcttccactttatgagaaaataatgagcttagctcctgacaagcttcatacccttatcgccagcggagatgtctatatacgctctgaaaaaccgttgcagtccatacctgaggccgacgttgtttgttacgggctgtgggtcgacccttccttggcgacgcaccatggggtattcgccagtgaccgcaaacatccggagcagctcgacttcatgctccagaagccttctttggcagaacttgaaagtctctctaagactcatcttttcctgatggacatagggatttggttattgtccgatcgcgcggtagagattttgatgaagcggtcccataaggagtcctctgaagaattgaaatactatgatctttacagcgactttgggcttgccctgggcacccacccacggatagaagatgaggaagtgaatactctctcagttgcaattctgcctctcccgggcggggagttttatcattacggtactagcaaagagctgatttcctccaccctgtcagtccagaataaggtttatgaccaacgccggatcatgcatagaaaggtcaaaccgaaccctgccatgtttgtccaaaatgcggtagtacgtatcccactgtgcgctgagaacgcagacctctggattgaaaactcacatataggtccgaagtggaagatagcgagccggcatattattactggggtgccggagaacgactggagtttagcagtgccagcaggggtatgtgtcgacgttgtcccgatgggtgataaaggttttgtggcacggccttacgggctggacgatgtatttaaaggagacctgagagattcaaaaaccactttgaccggaatcccattcggtgaatggatgtccaaacggggactctcctacactgatctgaaaggtcggactgacgaccttcaagccgtgagcgtgtttccgatggtcaattccgttgaggaattgggcctcgtactccgctggatgctttcagaacctgagttggaagagggtaagaacatctggcttcgctccgagcacttctctgcggatgaaataagcgctggagcgaacctgaagcgcctctatgctcaacgggaagaattccggaaggggaactggaaagcattagccgttaaccatgagaagtccgtattttaccaactcgatttggccgatgcggcggaggatttcgtacggttgggactcgacatgcctgaattactccctgaggacgccctgcagatgagtcgcatccataatagaatgcttcgcgctcgcatcctcaagctcgacggcaaagattaccggcctgaggaacaggcagcgttcgatctgctgcgcgacggtttgctcgatggaatctctaaccgtaaaagcacaccaaagctggacgtttattctgaccaaatagtgtggggccggagcccagtacggattgatatggccggcggctggaccgacaccccaccttatagcttatattccggaggcaatgttgtgaaccttgcgattgaattaaatggtcaacctccattgcaggtatacgttaagccgtgtaaggacttccatatcgtcctgcggtccattgacatgggggctatggagatagtctcaacttttgacgagctgcaagactacaaaaagatcggttccccgttttccataccaaaagccgctctctctcttgctggattcgccccggcatttagcgctgtgtcatatgcatcattggaggaacagttgaaagattttggagctggtattgaggttacgttattagccgccattccggctggttctggtcttggtacatcatcaatactggcttccacagtgctcggggcaattaatgatttttgtggactcgcatgggacaaaaatgaaatttgtcagagaacattagttttagagcaactgctgactacgggaggaggctggcaggaccagtacggcggggtcttgcaaggtgttaagcttctgcagaccgaggccgggttcgcgcaatccccattagttagatggctgccagaccacctctttacgcaccctgaatacaaggactgccatttgttatattacacaggaattacgcgtacggcaaagggaatactggcggagatcgtctctagcatgtttctcaatagtagcctgcacctgaatttactctcagaaatgaaagcgcatgccctcgacatgaacgaggctatacagcgcggctcctttgttgagttcggtcgccttgtcggaaagacatgggagcaaaacaaggcactcgacagtggtaccaacccaccagcagtcgaagccataatcgacttaattaaggattatacattagggtataaactcccaggggctggaggaggcgggtacctctacatggttgctaaagatccgcaggcagcagtgcgcatccgcaaaattttaacagagaacgcgccaaaccctcgtgcccggtttgtggaaatgactctgtccgacaaaggctttcaagtaagtcggtcctaa

SEQ ID NO: 7 - DNA, 900 bp, unidentified
  source 1..900: mol_type = other DNA
gcctttaaagtcgtccaaatctgcggcggacttggcaaccagatgttccagtatgccttcgccaagagccttcagaagcacagcaatacgccggtccttctggacatcacgtcctttgactggagcgatcgcaagatgcagctggaacttttcccgatcgatcttccatatgcctccgccaaagaaatcgccatcgccaagatgcagcaccttccgaagctggtccgcgatgctcttaagtgtatgggcttcgaccgcgtcagccaagaaatcgtcttcgagtacgaaccgaagcttctgaagccgagcagacttacgtacttcttcggctatttccaagatccgcgctatttcgacgccatttccccgcttatcaagcagacgttcacactgccgccaccgccggaaaacaacaagaacaataataagaaagaggaggagtaccagtgcaagctgagccttattcttgccgccaagaatagcgtcttcgtccacatcagacgcggagactatgtcggcatcggctgtcagctgggcatcgactatcagaagaaggctcttgagtacatggctaaacgcgtcccgaacatggagctgttcgtcttttgcgaggatctggagttcacgcagaatcttgatcttggatacccgttcatggatatgacgacgcgcgataaggaggaggaggcttactgggacatgcttctgatgcagagctgccagcacggcattatcgccaatagcacgtacagctggtgggccgcctatcttatcgagaacccagagaaaatcatcatcggcccgaagcactggctgttcggccacgagaacatcctttgcaaggagtgggtcaagatcgagtcccacttcgaggtcaagagccagaagtacaacgcctaa

SEQ ID NO: 8 - protein, 300 aa, unidentified
MAFKVVQICGGLGNQMFQYAFAKSLQKHSNTPVLLDITSFDWSDRKMQLELFPIDLPYASAKEIAIAKMQHLPKLVRDALKCMGFDRVSQEIVFEYEPKLLKPSRLTYFFGYFQDPRYFDAISPLIKQTFTLPPPPENNKNNNKKEEEYQCKLSLILAAKNSVFVHIRRGDYVGIGCQLGIDYQKKALEYMAKRVPNMELFVFCEDLEFTQNLDLGYPFMDMTTRDKEEEAYWDMLLMQSCQHGIIANSTYSWWAAYLIENPEKIIIGPKHWLFGHENILCKEWVKIESHFEVKSQKYNA

SEQ ID NO: 9 - DNA, 714 bp, unidentified
  source 1..714: mol_type = other DNA
atgtcaacaaaaaagaaaccgctgacacaagaacaactggaagatgcaagaagactgaaagcaatttatgaaaaaaagaaaaatgaactgggcctgtcacaagaatcagttgcagataaaatgggcatgggccaaagcggcgttggcgcactgtttaatggcattaatgcactgaatgcatataatgcagcactgctgacaaaaattctgaaagtttcagttgaagaattttcaccgtcaattgcaagagaaatttatgaaatgtatgaagcagtttcaatgcaaccgtcactgagatcagaatatgaatatccggttttttcacatgttcaagcgggcatgttttcaccgaaactgagaacatttacaaaaggcgatgcagaaagatgggtttcaacaacaaaaaaagcatcagattcagcattttggctggaagttgaaggcaattcaatgacagcaccgacgggctcaaaaccgtcatttccggatggcatgctgattctggttgatccggaacaagcagttgaaccgggcgatttttgcattgcaagactgggcggcgatgaatttacatttaaaaaactgattagagatagcggccaagtttttctgcaaccgctgaatccgcaatatccgatgattccgtgcaatgaatcatgctcagttgttggcaaagttattgcatcacaatggccggaagaaacatttggctaa

SEQ ID NO: 10 - DNA, 45 bp, synthetic construct
  source 1..45: mol_type = other DNA
aatctactggtattgtacgtatggttattgcatcagttgaagatg

SEQ ID NO: 11 - DNA, 50 bp, synthetic construct
  source 1..50: mol_type = other DNA
tgtgttgaaatatgttttgttgatgctttttttgtaattgtatatgttgt

SEQ ID NO: 12 - DNA, 49 bp, synthetic construct
  source 1..49: mol_type = other DNA
acgtacaataccagtagatttcataaacattttttttcctccttttctc

SEQ ID NO: 13 - DNA, 47 bp, synthetic construct
  source 1..47: mol_type = other DNA
acaaaacatatttcaacacaatacaaatgggttagttaaaaaagcag

SEQ ID NO: 14 - DNA, 73 bp, synthetic construct
  source 1..73: mol_type = other DNA
cgcattatccgcattaacgacgatgatgttttccgcctcggctctcgccatggttattgcatcagttgaagat

SEQ ID NO: 15 - DNA, 77 bp, synthetic construct
  source 1..77: mol_type = other DNA
gtcgttaatgcggataatgcgaggatgcgtgcacctgtttttattttcatggtatatctccttgtgtgagttaatct

SEQ ID NO: 16 - DNA, 37 bp, synthetic construct
  source 1..37: mol_type = other DNA
taaggagatataatgagcaaaggagaagaacttttca

SEQ ID NO: 17 - DNA, 36 bp, synthetic construct
  source 1..36: mol_type = other DNA
ggtggcagcagttatttgtagagctcatccatgcca

SEQ ID NO: 18 - DNA, 27 bp, synthetic construct
  source 1..27: mol_type = other DNA
tacaaataactgctgccaccgctgagc

SEQ ID NO: 19 - DNA, 52 bp, synthetic construct
  source 1..52: mol_type = other DNA
tcctttgctcattatatctccttattaaagttaaacaaaattatttctacag

SEQ ID NO: 20 - DNA, 48 bp, synthetic construct
  source 1..48: mol_type = other DNA
caacgcctaatttgtttaactttaagaaggagatataccatgcaaaag

SEQ ID NO: 21 - DNA, 39 bp, synthetic construct
  source 1..39: mol_type = other DNA
accagatcaatacaaataataaacgcatgagaaagcccc

SEQ ID NO: 22 - DNA, 41 bp, synthetic construct
  source 1..41: mol_type = other DNA
tgagtgagctaacttacttgacaattaatcatccggctcgt

SEQ ID NO: 23 - DNA, 44 bp, synthetic construct
  source 1..44: mol_type = other DNA
gttaaacaaattaggcgttgtacttctggctcttgacctcgaag

SEQ ID NO: 24 - DNA, 33 bp, synthetic construct
  source 1..33: mol_type = other DNA
tgtattgatctggtacacggtcacactgcttcc

SEQ ID NO: 25 - DNA, 47 bp, synthetic construct
  source 1..47: mol_type = other DNA
gtaagttagctcactcattaggcaccgggatctcgaccgatgccctt

SEQ ID NO: 26 - DNA, 50 bp, synthetic construct
  source 1..50: mol_type = other DNA
ggctgttttggcggatgagagaagattttcagcctgatacagattaaatc

SEQ ID NO: 27 - DNA, 57 bp, synthetic construct
  source 1..57: mol_type = other DNA
gctgtttcctgtgtgacctccttaatttttaaccaatgcttcgtttcgtatcacaca

SEQ ID NO: 28 - DNA, 58 bp, synthetic construct
  source 1..58: mol_type = other DNA
ggttaaaaattaaggaggtcacacaggaaacagctatgaccatgattacggattcact

SEQ ID NO: 29 - DNA, 47 bp, synthetic construct
  source 1..47: mol_type = other DNA
ctctcatccgccaaaacagccaattatttttgacaccagaccaactg

SEQ ID NO: 30 - DNA, 39 bp, synthetic construct
  source 1..39: mol_type = other DNA
ccatcatcaccactaagatccggctgctaacaaagcccg

SEQ ID NO: 31 - DNA, 51 bp, synthetic construct
  source 1..51: mol_type = other DNA
ggtatatctccttcttaaagttaaacaaaattatttctagaggggaattgt

SEQ ID NO: 32 - DNA, 44 bp, synthetic construct
  source 1..44: mol_type = other DNA
ctttaagaaggagatataccatgtcccctatactaggttattgg

SEQ ID NO: 33 - DNA, 37 bp, synthetic construct
  source 1..37: mol_type = other DNA
cacgtttggtggtggcgaccatcctccaaaaatggcc

SEQ ID NO: 34 - DNA, 40 bp, synthetic construct
  source 1..40: mol_type = other DNA
cgaccatcctccaaaaatggcctttaaagtcgtccaaatc

SEQ ID NO: 35 - DNA, 51 bp, synthetic construct
  source 1..51: mol_type = other DNA
ggatcttagtggtgatgatggtgatgggcgttgtacttctggctcttgacc